(19) United States
(12) Patent Application Publication
(10) Pub. No.: US 2010/0130871 A1
(43) Pub. Date: May 27, 2010

(54) SPECTRAL IMAGING DEVICE FOR HIRSCHSPRUNG'S DISEASE

(75) Inventors: Philip K. Frykman, Los Angeles, CA (US); Daniel L. Farkas, Los Angeles, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/594,803

(22) PCT Filed: Apr. 2, 2008
(86) PCT No.: PCT/US08/59150

§ 371 Date: Oct. 5, 2009

Related U.S. Application Data
(60) Provisional application No. 60/910,508, filed on Apr. 6, 2007.

(51) Int. Cl. A61B 1/04; G06K 9/00

(52) U.S. Cl. 600/477; 382/128; 600/407

(57) ABSTRACT

The subject matter disclosed herein relates to the field of spectral imaging in the diagnosis and treatment of Hirschsprung's disease. Devices and methods are provided that enhance and accurately diagnose Hirschsprung's disease intraoperatively using spectral imaging technology.

### GOVERNMENT RIGHTS

The subject matter described herein may have been supported in part by Grant No. 1435-04-04-GT-43096 awarded by the U.S. Navy to Cedars-Sinai Medical Center's Minimally Invasive Surgical Technologies Institute. The government may have certain rights in this subject matter.

### FIELD OF THE SUBJECT MATTER

The present subject matter relates to a spectral imaging device and methods of using the spectral imaging device and methods in the diagnosis and treatment of Hirschsprung's disease.

### BACKGROUND OF THE SUBJECT MATTER

All publications herein are incorporated by reference to the same extent as if each individual publication or patent application was specifically and individually indicated to be incorporated by reference. The following description includes information that may be useful in understanding the present subject matter. It is not an admission that any of the information provided herein is prior art or relevant to the presently claimed subject matter, or that any publication specifically or implicitly referenced is prior art.

Hirschsprung's disease is the congenital absence of specialized nerve cells, i.e., ganglion cells, primarily affecting the lower portion of colon. When Hirschsprung's disease is untreated, it causes severe constipation that can lead to massive dilatation of the colon (megacolon), colonic obstruction, often leading to death by overwhelming infection. It affects approximately 1 in 5000 live births (J. Amiel and S. Lyonnet, “Hirschsprung disease, associated syndromes, and genetics: a review”, JMed Genetics 38:729-39[2001]), however, approximately 90% of Hirschsprung's patients are diagnosed in infancy and undergo surgical therapy early in life.

The current surgical therapy for Hirschsprung's disease consists of a minimally invasive endorectal pull-through procedure in the first month of life (K. E. Georgeson, R. D. Cohen, A. Hebra, et al, “Primary laparoscopic-assisted endorectal colon pull-through for Hirschsprung's disease: a new gold standard”, Ann Surg 229:678-682[1999]). The pull-through procedure consists of several steps, the most crucial of which involves the identification of the level at which ganglion cells are present in the colon. The initial step in the pull-through procedure is to identify this level of transition between ganglionic and aganglionic colon. This is routinely done by laparoscopically procuring multiple small biopsies of the colon wall which are then sent for rapid frozen sections to pathology. The pathologist looks for the presence or absence of ganglion cells (and other features) to determine the diagnosis in each specimen, thereby determining the point where the transition from normal to aganglionic colon occurs. The precise point of transition is absolutely critical to successfully performing the next stage of the surgery. The aganglionic colon is then removed and the normal colon is pulled through the anus and sutured in place. This surgery avoids the need for a colostomy and has proven to be safe and effective.

A critical stage of this procedure is the accurate and precise determination of the transition point from normal to aganglionic colon. This portion of the surgery typically takes approximately 60 minutes, during which time minimal operating is achieve. Most of this time is exhausted waiting for results of the biopsies determined by frozen section while the patient remains under general anesthesia. The cost of the operating room time is significant, and can amount to approximately $60 per minute (D. E. Beck, M. A. Ferguson, F. G. Opelka, J. W. Fleshman, P. Gervaz and S. D. Wexner, “Effect of Previous Surgery on Abdominal Opening Time”, Dis Colon Rectum, 43(12):1749-1753 [2000]; C. C. Cothren, E. E. Moore, J. L. Johnson, J. B. Moore, D. J. Ciesla and J. M. Burch, “Can we afford to do laparoscopic appendectomy in an academic hospital?”, The American Journal of Surgery, 196(6):973-977 [2005]; J. W. Haller, T. C. Ryken, T. A. Gallagher and M. W. Vannier, “Infrastructure for Image Guided Surgery”, at http://www.radiology.uiowa.edu/NEWS/Haller-PDF.pdf). Thus, waiting for the results of the biopsies of frozen sections can cost a patient upwards of $4000 during this stagnant period. In addition, incidents and probability of complication are increased with additional time under general anesthesia.

Despite the pathologists' expertise in reading rapid frozen sections, due to limited analysis time and human error, they are not always accurate in determining the point of aganglionosis in Hirschsprung's disease. On occasion the transition point from ganglionic to aganglionic colon is not accurately determined and a patient might have more or less colon removed than is appropriate. If too little colon is removed, thereby leaving aganglionic colon, the patient would likely develop significant constipation, which could potentially require an additional surgery for removal of the superfluous aganglionic colon. Conversely, if too much colon is removed, the patient would have increased stool frequency (diarrhea) which can result in body salt and mineral imbalances, dehydration, and skin breakdown.

As minimally invasive surgical techniques are widely accepted now and more and more microscopic and endoscopic procedures and devices have been adopted for surgery, a promising technique for diagnosis of Hirschsprung's disease may include incorporation of multi-spectral imaging.

Multi-spectral imaging provides digital images of a scene or object at a large, usually sequential number of wavelengths, generating precise optical spectra at every pixel. A spectral signature can be developed, that is, a quantitative plot of optical property variations as a function of wavelengths to help identify ganglionic and aganglionic tissue. However, multi-spectral images constitute a particular class of images that require specialized coding algorithms. In multi-spectral images, the same spatial region is captured multiple times using different imaging modalities. These modalities often consist of measurements at different optical wavelengths. The term “spectral signature” is used differently in various science fields. Here, it is another name for a plot of the variations in absorbed, reflected or emitted light intensity as function of wavelengths. These signatures are useful for identifying and separating materials or objects of interest, and can be used in connection with Hirschsprung's disease to represent differing tissue characteristics in medical imaging.

However, limitations exist in the current art that prevent the best possible use of multi-spectral imaging in the operating room. For example, methods to obtain useable information real-time in the operation room would be ideal, and clinicians are unlikely to be interested in adoption of spectral imaging if more than a few minutes elapse from acquisition to display.

Current multi-spectral tools do not meet these requirements because of the large amounts of processing required. Processing currently present a major bottleneck, as it typically requires manual signature selection. In manual signature selection, clinicians choose an appropriate reference signature from the image cube and if the signature selection was not good enough, the classification and visualization of the image would be unsatisfactory.

Accordingly, there is a need in the art for a rapid, less invasive, and more accurate technique for diagnosis of Hirschsprung's disease by distinguishing aganglionic cells from ganglionic cells using spectral imaging technology. To address this technique, there is also a need in the art to develop a device that can perform this spectral imaging function and create real-time, or near real-time, images with appropriate differentiation methods to aid in identifying normal and abnormal tissue, rapidly and accurately, all within the surgery setting. Further attributes of the device would include an intelligent, automated process for signature selection. The development of this technique and device will at a minimum, improve on the current method of diagnosis, improve patient treatment with less time under anesthesia, and significantly improve the accuracy of differentiation between normal and abnormal tissue in the diagnosis and treatment of Hirschsprung's disease. In addition, the improvements achieved above will lead to a substantial reduction of medical costs and risks associated with the treatment of Hirschsprung's disease.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 illustrates a cross section of the intestine and the extralumenal application of the device according to one embodiment of the present invention.

FIG. 2 illustrates a cross-section of the abdomen and the application of the laparoscopic adapted device according to one embodiment of the present invention.

FIG. 3 graphically illustrates the spectral statistics associated with the experiments in Example 1.

FIG. 4 graphically illustrates the spectral signatures ascertained from the experiments in Example 1. The term spectral signature as used herein refers to a quantitative plot of optical property variations as a function of wavelengths.

FIG. 5 is a chart illustrating the number of spectral signatures acquired from the experiments in Example 1.

FIG. 6 is a chart illustrating the results of spectral signature imaging based upon the analytical model in Example 3.

FIG. 7 illustrates a flow chart of an automatic signature detection process according to one embodiment of the invention.

FIG. 8 illustrates a block diagram of the components of the exemplary spectral image analysis system.

### DETAILED DESCRIPTION OF THE SUBJECT MATTER

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present subject matter. Indeed, the present subject matter is in no way limited to the methods and materials described. For purposes of the present subject matter, the following terms are defined below.

“Aganglionic” as used herein refers to Ganglionic cells in the colon, which have lost their ability to function as nerve cells.

“Extralumenally” as used herein refers to the attachment and/or use of a device outside the lumen.

“Frozen Section” or “Frozen Sections” refers to the technique allowing examination of histologic sections of the colon specimen(s) removed from the patient.

“Ganglionic” as used herein refers to specialized nerve cells in the colon.

“Intralumenally” as used herein refers to the attachment and/or use of a device inside the walls of the lumen.

“Intraoperatively” as used herein refers to treatment of the colon while in surgery.

“Device” as used herein refers to materials disclosed and inferred herein relating to the subject matter, spectral imaging device and methods for the diagnosis and treatment of Hirschsprung's disease.

“Real-Time” refers to the response to signals and/or events immediately or within a short period of time.

“Spectral Signature” as used herein refers to a quantitative plot of optical property variations in absorbed, reflected or emitted light intensity as a function of wavelength, time and other possible scale units for each pixel in the image depending on the optical imaging modes, herein specifically associated with aganglionic and ganglionic colon.

“Transition Point” refers to the level or plane in the colon at which specialized nerve cells (ganglion cells) transition to aganglionic cells.

“Treatment” and “treating” as used herein refer to both therapeutic treatment and prophylactic or preventative measures, wherein the object is to prevent, slow down and/or lessen the disease even if the treatment is ultimately unsuccessful.

The present subject matter is directed to a spectral imaging device and methods of using the device in the diagnosis and treatment of Hirschsprung's disease. The spectral imaging device uses novel image acquisition, processing, and analysis, which may be utilized intraoperatively and can accurately and precisely distinguish normal from aganglionic colon in patients with Hirschsprung's disease. Furthermore, the device allows for an advantageous surgical technique that is faster than current methods used to detect diseased tissue, is less invasive, and more accurate. It will also allow for decreased operative time and increased medical accuracy, thereby improving on the current devices and methods practiced and potentially making treatment less expensive and safer. For example, the device will obviate the need for biopsy and will be more accurate than visual assessment of biopsied tissue.

The method of using the device to diagnose and treat Hirschsprung's disease will involve using the device to intraoperatively look for the presence or absence of ganglion cells (and other features), to determine the level where the transition from normal to aganglionic colon occurs, and accomplished both tasks in real-time. The precise level of the transition point is critical to diagnosing the exact location of the diseased tissue and treating the disease by performing surgery to completely remove the aganglionic tissue. Detection of the transition point using the device can be by visual image output from the device, whereby results may be indicated according to a certain color code (e.g., green=normal tissue, red=cancerous tissue, blue=inflammation). Real-time, or near real-time, images with appropriate color allocation to differentiate between normal and abnormal tissue are created using the device, and greatly enhance the diagnosis and treatment of Hirschsprung's disease. The device can also output results by any number of alternative mechanisms, including, but not limited to, sound clues, graphical tools, and diagnostic printouts. In surgery, the aganglionic colon is removed and the normal colon is pulled through the anus and sutured in place and it is imperative that the transition point between aganglionic and normal tissue is detectable.

In one embodiment the device can be directed on the serosal surface (outside wall) of the colon, extralumenally, or the inside wall of the colon, endolumenally, and can be used in open surgery or laparoscopic (minimally invasive) surgery.

In a further embodiment, the inventive spectral imaging device overcomes the prior art pull-through techniques for treating Hirschsprung's Disease reliance on intraoperative frozen sections to determine the plane of aganglionosis. Intraoperative removal of sections of the colon requires that a pathologist read the specimen to determine whether tissue is aganglionic, or whether the tissue is healthy or ganglionic. Use of the inventive spectral imaging device significantly reduces the possibility of tainted specimens or inadvertent errors in the pathologist's interpretation of the frozen sections. The spectral imaging device also increases the efficiency in identifying the aganglionic colon in that the device can distinguish between normal and aganglionic tissue in vivo during a surgical procedure. The inventive spectral imaging device is critical to a successful and seamless procedure for diagnosing and treating Hirschsprung's disease.

In another embodiment the spectral imaging device is developed for in vivo use for real-time intraoperative localization of the transition point based upon a spectral signature algorithm generated from data obtained from patients having Hirschsprung's Disease. Spectral sampling of the regions of interest can be performed, analyzed, and read out according to the methods described in Examples 1 and 3.

Spectral signature management, image processing, visualization and dimensionality algorithm between normal and agangliotic colon tissue may be performed using SpectralJ software, a custom plug-in which may developed for the very popular open source digital image processing tool called ImageJ (available at http://rsb.info.nih.gov/ij/ or National Institute of Health, Bethesda, Md.). After converting 3-D spectral dataset into a series of TIFF format images, ImageJ may import the images as an image stack. SpectralJ can use this information stored in the database.

Hypersonic SQL, a well known open source Java database management software, maybe used to integrate database functionalities in the SpectralJ plug-in software. Using the database library, spectral signatures can register, loaded and searched in and from a local computer or via the internet.

Spectral matching is measured by an algorithm using spectral similarity to evaluate the difference between a reference spectral signature chosen by user, or automatically detected and the target pixel spectral signature in the image. Spectral Angle Mapper (SAM), a popular calculation algorithm, may be used to measure spectral similarity measures in hyperspectral image analysis. SAM calculates the angle between two spectra and uses it as a measure of discrimination (J. Schwarz J. and K. Staenz, “Adaptive Threshold for Spectral Matching of Hyperspectral Data”, Canadian Journal of Remote Sensing, 27(3): 216-224 [2001]).

It yet another embodiment, the device involves an endoscopic catheter that can be inserted through a 3 mm or standard laparoscopic port. The endoscopic catheter of the device shines full spectrum visible wavelengths of light (350 to 710 nm) and collects and analyzes the light reflected back from the tissue. It should be manufactured in full compliance with the FDA's GMP and ISO 9001 regulations. The light source to which the scope is connected is based on a 150 W xenon lamp, similar to that used in conventional endoscope light sources. The energy delivered is less than 5 milliwatts. A fast, sensitive CCD camera mounted to the endoscope, a modified laptop personal computer, and control electronics can also be part of the overall device. Detection of the transition point using the device can be by real-time, or near real-time, visual image output from the device whereby results are indicated according to a certain color code (e.g., green=normal tissue, red=cancerous tissue, blue=inflammation). Real-time images with appropriate color allocation to differentiate between normal and aganglionic tissue or other results indicators, including, but not limited to, sound clues, graphical tools, and diagnostic printouts are contemplated.

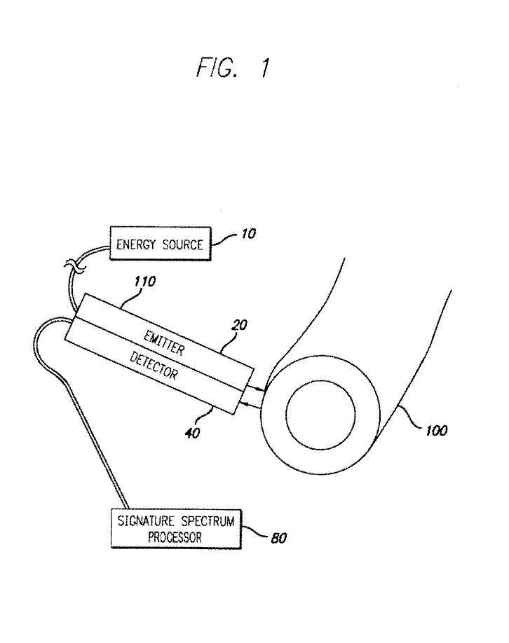

According to another embodiment, FIG. 1 shows the device and use of the device outside of the lumen 100, or extralumenally. The endoscopic probe 110 is comprised of an emitter 20 (infrared (IR), variable, or ultraviolet (UV)) and a detector 40. The emitter 20 shines full-spectrum visible wavelengths of light and is connected to an energy source 10 for generating the emitter signal. The detector 40 collects and analyzes the light reflected back from the tissue and relays the information to the signature spectrum processor 80, whereby a signature spectrum is detected for aganglionic versus normal intestinal tissue. Detection of a specific signature spectrum may be based upon an algorithm developed from a collection of spectral images of normal and aganglionic colon. The device may also be used inside the lumen 100, or “intralumenally,” using an endoscopic probe 110 through an endoscope. The device may further comprise a CCD camera mounted to the endoscope, a modified computer, and control electronics.

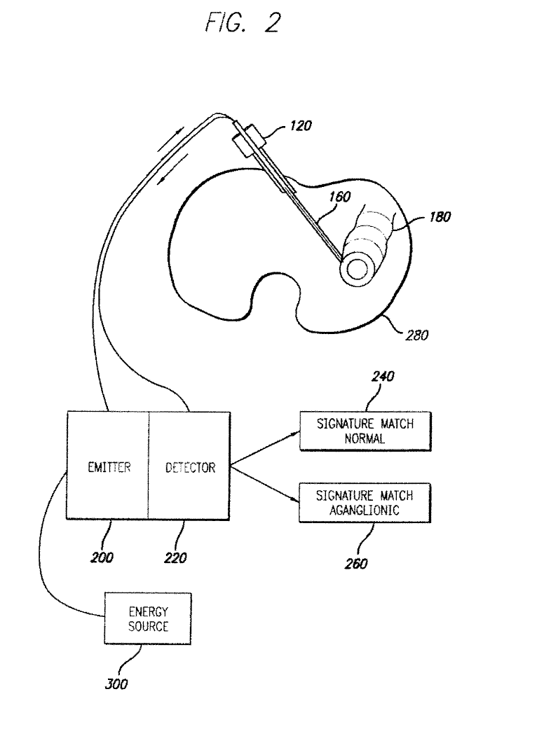

According to another embodiment, FIG. 2 shows a cross-section of an abdomen 280 with the device comprised of a laparoscopic adapted emitter-detector probe 160 that is connected to an emitter 200 and a detector 220 whereby the emitter shines light onto the tissue and the detector collects and analyzes the light reflected back from the tissue. An energy source 300 is connected to the emitter 200 for generating the emitter signal. The probe is shown placed through a laparoscopic port 120, whereby a signature match 240, 260 is detected for aganglionic 260 or normal, ganglionic 240 tissue. Detection of a specific match may be based upon an algorithm developed from a collection of spectral images of normal and aganglionic colon. One way to develop an algorithm is to collect spectral images, sample the spectral images at certain wavelengths and then determine a ratio of intensities. The device may be used inside the lumen 180, intralumenally, or outside the lumen 180, extralumenally. The device may further comprise a CCD camera mounted to the endoscope, a modified computer, and control electronics.

In yet another embodiment the device may acquire spectral images based on acousto-optic tunable filters (AOTF). AOTF is a solid-state, electronically tunable, frequency-agile, optical band-pass filter. It may consist of a piezoelectric transducer affixed to an optical quality crystal. Radio frequencies applied to the crystal can be used to filter single wavelengths of light from a broadband light source. It is capable of performing real-time spectral signature analysis and thus may be useful for in vivo applications. The device is adapted for in vivo use and may acquire spectral images of the intestinal tissue based on AOTF. An imaging system, or similar systems, according to U.S. Pat. No. 5,796,512 or U.S. Pat. No. 5,841,577 may be used. The device utilizing AOTF can be used in vivo either extralumenally or intralumenally by way of an endoscopic port, and may also comprise a light source, an energy source, a CCD camera mounted to the endoscope, a modified computer, and control electronics.

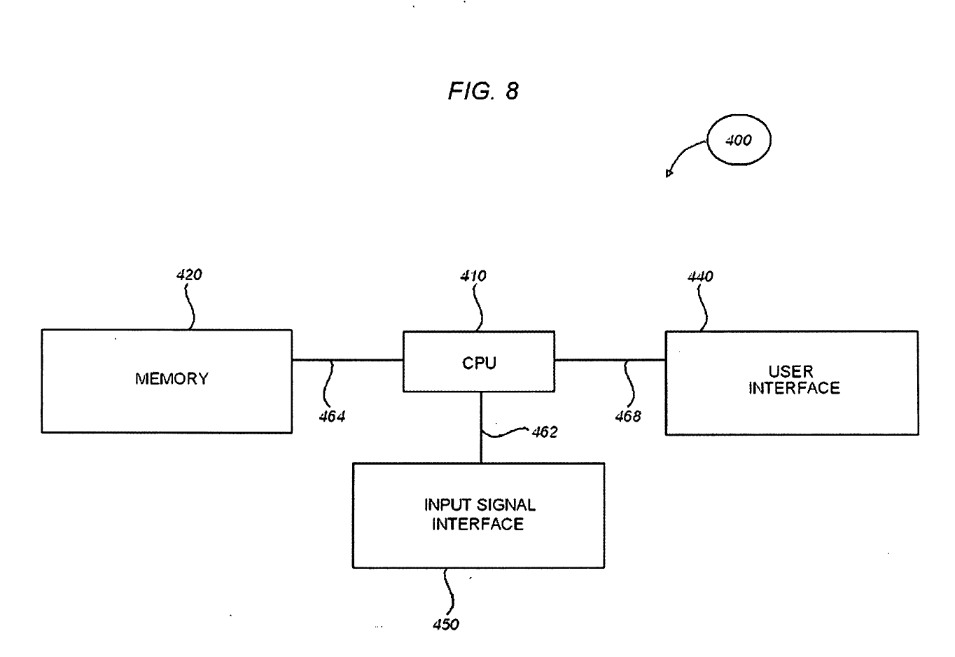

In another embodiment the device may be coupled to a spectral image analysis system (System) for analysis of the multi spectral image. FIG. 8 is a block diagram of the components of the exemplary system 400. The system 400 may include a programmable central processing unit (CPU) 410 which may be implemented by any known technology, such as a microprocessor, microcontroller, application-specific integrated circuit (ASIC), digital signal processor (DSP), or the like. The CPU 410 may be integrated into an electrical circuit, such as a conventional circuit board, that supplies power to the CPU 410. The CPU 410 may include internal memory or memory 420 may be coupled thereto. The memory 420 may be coupled to the CPU 410 by an internal bus 464.

The memory 420 may comprise random access memory (RAM) and read-only memory (ROM). The memory 420 contains instructions and data that control the operation of the CPU 410. The memory 420 may also include a basic input/output system (BIOS), which contains the basic routines that help transfer information between elements within the system 400. The present subject matter is not limited by the specific hardware component(s) used to implement the CPU 410 or memory 420 components of the system 400.

Optionally, the memory 420 may include external or removable memory devices such as floppy disk drives and optical storage devices (e.g., CD-ROM, R/W CD-ROM, DVD, and the like). The system 400 may also include one or more I/O interfaces (not shown) such as a serial interface (e.g., RS-232, RS-432, and the like), an IEEE-488 interface, a universal serial bus (USB) interface, a parallel interface, and the like, for the communication with removable memory devices such as flash memory drives, external floppy disk drives, and the like.

The system 400 may also include a user interface 440 such as a standard computer monitor, LCD, colored lights, or other visual display including a bedside display. In one embodiment, a monitor or handheld LCD display may provide an image of a colon and a visual representation of the estimated location between normal and aganglionic colon tissue. The user interface 440 may also include an audio system capable of playing an audible signal.

The user interface 440 may permit the user to enter control commands into the system 400. For example, the user could command the system to store information such as the spectral signature values of the colon tissue. The user interface 440 may also allow the user or operator to enter patient information and/or annotate the data displayed by user interface 440 and/or stored in memory 420 by the CPU 410. The user interface 440 may include a standard keyboard, mouse, track ball, buttons, touch sensitive screen, wireless user input device, and the like. The user interface 440 may be coupled to the CPU 410 by an internal bus 468.

Optionally, the system 400 may also include an antenna or other signal receiving device such as an optical sensor for receiving a command signal such as a radio frequency (RF) or optical signal from a wireless user interface device such as a remote control. The system 400 may also include software components for interpreting the command signal and executing control commands included in the command signal. The system may also include software and hardware component for access to worldwide electronic networks. These software components may be stored in memory 420.

The system 400 includes an input signal interface 450 for receiving the multi spectral image and associated signals. The input signal interface 450 may include any standard electrical interface known in the art for connecting a double dipole lead wire to a conventional circuit board as well as any components capable of communicating a low voltage time varying signal from a pair of wires through an internal bus 462 to the CPU 410. The input signal interface 450 may include hardware components such as memory as well as standard signal processing components such as an analog to digital converter, amplifiers, filters, and the like.

For in vivo use, the device may be mobile and may utilize fiber optic cables for transmitting endoscope input and output.

Use of the spectral imaging device is a rapid and accurate way for determining the transition point in the colon between aganglionic and ganglionic tissue of individuals suffering from Hirschsprung's Disease. Accordingly, in another embodiment, a method for diagnosing and treating Hirschsprung's disease is performed using the inventive spectral imaging device. According to the method, treatment will proceed faster and with more accuracy than prior art methods, thereby perfecting determination of the transition point between diseased and normal tissue and optimizing accuracy of pull-through procedures.

Multi-spectral optical imaging is promising because minimally invasive surgical techniques are widely accepted and more and more micro-endoscopic procedures and devices are adopted. Moreover, there exists no direct visualization method to discriminate between normal and abnormal tissues except naked eye assessment of the operating field. In order to be useful in a surgical environment, imaging-based diagnosis needs to be nearly real-time, with enough discrimination power from the displayed result image. Moreover, it should be easy to use and require no extra procedures. Therefore, spectral imaging via the device that provides automatic data acquisition and display is crucial to the performance of rapid and accurate diagnosis of the point of aganglionosis in Hirschsprung's disease and treatment of the disease by removal of the proper amount of aganglionic tissue.

Hirschsprung's disease is a good application area for spectral imaging-based diagnosis because the spectral signatures between normal and aganglionic segments are highly reproducible and relatively large. Moreover, the effectiveness and the benefit of this kind of “optical biopsy” are potentially quite significant, promising faster, more quantitative (and thus more objective) tissue assessment in vivo, for intrasurgical navigation and decision-making.

The above disclosure generally describes the present subject matter, and all patents and patent applications, as well as publications, cited in this disclosure are expressly incorporated by reference herein. A more complete understanding can be obtained by reference to the following Examples, which are provided for purposes of illustration only and are not intended to limit the scope of the subject matter.

### EXAMPLES

The following examples describe a range of applications of the device and methods of the present subject matter, as well as a number of components that may be readily integrated and/or otherwise used in connection with the same. These Examples demonstrate some of the many configurations of the device of the subject matter, the uses of the device, and the potential impact it may have on the conventional practice of medicine. Modifications of these examples will be readily apparent to those skilled in the art who seek to treat patients whose condition differs from those described herein.

### Example 1

Preclinical studies using spectral imaging techniques are performed on a mouse model of Hirschsprung's Disease. The mouse model is named piebald-lethal. This mutant mouse strain is born with aganglionosis of the distal colon and then will develop abnormal dilation of the colon, i.e., megacolon, thereby leading to death around one month of age (K. Hosoda, R. E. Hammer, J. A. Richardson, A. G. Baynash, J. C. Cheung, A. Giaid, M. Yanagisawa, “Targeted and natural (piebald-lethal) mutations of endothelin-B receptor gene produce megacolon associated with spotted coat color in mice”, Cell 79:1267-76[1994]; E. P. Nadler, P. Boyle, A. D. Murdock, C. Dilorenzo, E. M. Barksdale, H. R. Ford, “Newborn endothelin receptor type B mutant (piebald) mice have a higher resting anal sphincter pressure than newborn C57BL/6 mice”, Contemp Top Lab Anim Sci 42:36-8[2003]). A total of six (6) piebald-lethal mice are used, three (3) homozygote (sl/sl) and three (3) heterozygote (+/sl). The (+/sl) animals serve as the control animal. The (sl/sl) animals have both distal colon (aganglionic) and proximal colon (normal) imaged. The proximal colon will also serve as an internal control. Spectral imaging can be achieved by Fourier transform microinterferometry with an imaging set-up consisting of a Nikon E800 microscope, a Xenon arc lamp, a CCD camera, and imaging interferometer and imaging software provided by Applied Spectral Imaging, Inc.

Mice are given isoflurane inhalational anesthesia, placed on a warmed platform, and laparotomy is performed. The colon is exposed and grasped in an atraumatic clamp. In vivo spectral imaging of the serosal surface of the lower colon is performed in both animal groups. Light collected is the reflected image, and crossed-polarizers are employed to mitigate the surface-only reflections from the sample. The reflected light is channeled through an interferometer to a CCD camera.

Imaged segments are marked at the site of imaging for pathologic evaluation using standard methods.

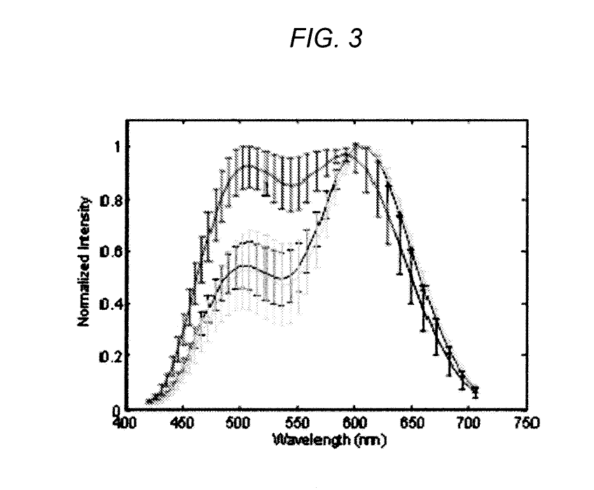

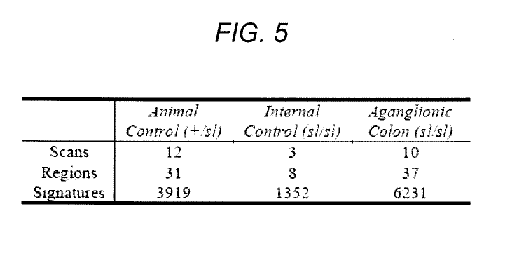

From the six mice for the experiment on Hirschsprung's disease, 15 control images from animal control and internal control from proximal colon of homozygous mice were scanned and 10 aganglionic colon images were acquired. Total signatures analyzed were 3919, 1352, and 6231 from animal control, internal control, and aganglionic colon, respectively (see FIG. 5). FIG. 3 illustrates the spectral statistics associated with the experiment resulting from performance of pixel spectral analysis. The spectral curves are generated with standard deviation bars. The bottom most curve at 450 nm is the curve generated based upon analysis of the homozygous mouse internal control (proximal colon). The curve just adjacent to this curve is the curve generated based upon analysis of the heterozygous animal control. The top-most curve at 450 nm is the spectral curve generated based upon analysis of the homozygous mouse aganglionic (distal colon). The animal control and the internal control spectral curves are nearly superimposed on each other and the curve generated based upon analysis of the aganglionic portion of the colon is markedly different. The signature acquired is clearly different between aganglionic colon and normal colon after peak-normalization. The peak values at 500 nm were almost as strong as 600 nm in aganglionic colon, but the values in normal colon were almost half of the values at 620 nm (see FIG. 3).

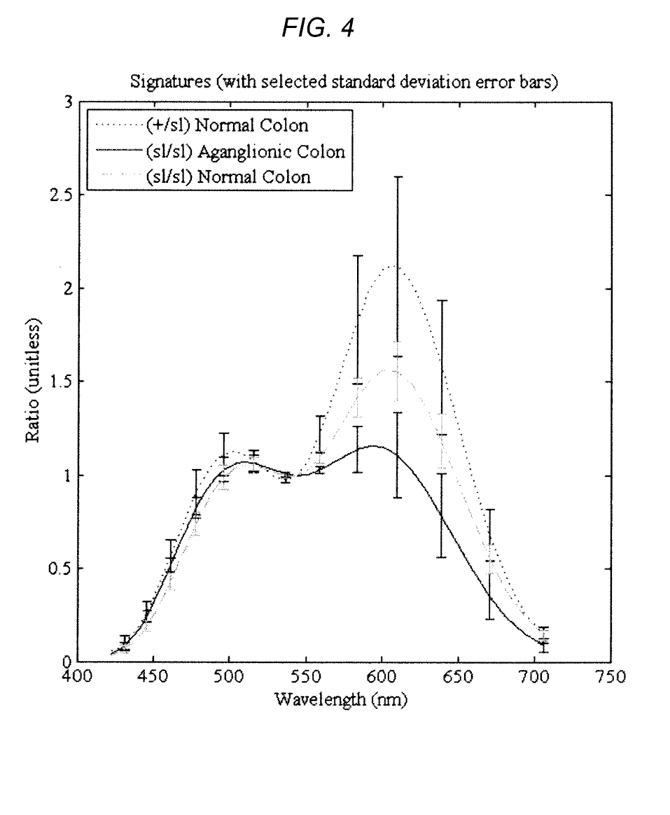

The spectral signatures comparing (sl/sl) normal colon to (sl/sl) aganglionic colon and (+/sl) normal colon are clearly distinguishable from one another. FIG. 4 shows the signatures with selected standard deviation error bars with curves generated by plotting a unitless ratio against wavelength. The ratios of the curves at 609 nm and a ratio of 1.47 demonstrate that an inventive spectral imaging device implementing data based upon consistent differences in the spectral signatures and algorithms developed therefrom can distinguish normal from aganglionic colon with high sensitivity and specificity. A ratio of 1.47 based on the average differences after normalization at the wavelength 609 nm shows sensitivity 97%, specificity 94%, positive predictive value (PPV) 92%, and negative predictive value (NPV) 98% (P. K. Frykman, M. Gaon, E. Lindsley, J. Lechago, A. P. Chung, Y. Xiong and D. L Farkas, “A Novel, Rapid, and Accurate Method for Determining the Level of Aganglionosis in Hirschsprung's Disease Using Spectral Imaging”, Page 76, Proceeding of IPEG 2006).

Based upon these results, an algorithm to calculate the ratio or intensities at the reported wavelengths can be created, thereby developing a spectral signature for normal versus aganglionic colon. Based upon the spectral signature algorithm, spectral sampling and analysis can be performed quickly, for example, in less than one (1) second. A similar device and spectral signature can be developed for an in vivo spectral imaging device for real-time intraoperative localization of the transition point.

### Example 2

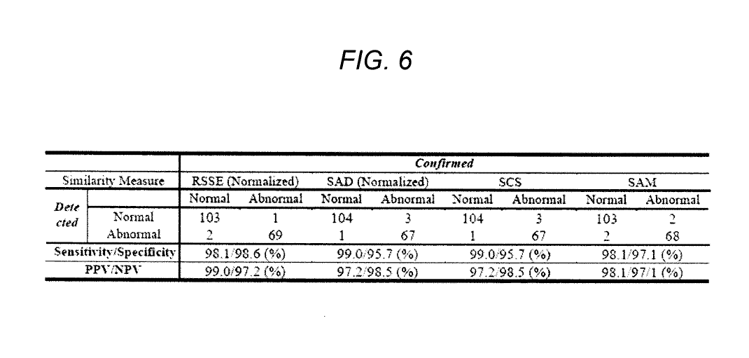

A second method for analyzing the results obtained in Example 1 to arrive at an intelligent spectral signature for use in the novel in vivo spectral imaging device is performed herein. The end results obtained using this alternative method are similar to the results achieved in the analysis of Example 1. Discrimination between normal and aganglionic segments of the colon was possible with over 95% sensitivity and specificity (see FIG. 6).

The intelligent spectral signature imaging analysis according to this Example provides automatic signature selection based on machine learning algorithms and database search-based automatic color allocations, and selected visualization schemes matching these approaches.

A spectral signature analysis is performed between the normal and aganglionic colon tissue collected by spectral imaging in Example 1. The signature analysis is performed by software called “SpectralJ software”, a custom plug-in that can be developed for an open source digital image processing program ImageJ (available at http://rsb.info.nih.gov/ij/ or National Institute of Health, Bethesda, Md.). A spectral data cube can be converted into a series of TIFF format images and then can be imported by ImageJ. SpectralJ can use this information stored in the database.

Hypersonic SQL, an open source java database management software known to those having skill in the relevant art, can be used to integrate database functionalities in the SpectralJ plug-in software. Using the database library, spectral signatures can be registered, loaded and searched in and from a local computer or via internet.

An automatic signature detection feature can be implemented based on the K-means algorithm, a clustering method. Clustering is a data mining algorithm for unsupervised learning or indirect knowledge discovery. Many data mining methods develop models that predict how to classify new data from classified training data sets. Classified training data sets and discrimination between independent and dependent variables are not needed in clustering algorithms. Instead, assuming similar data records will act similarly, the data will be found in the same group, i.e., “cluster” (T. Hastie, R. Tibshirani and J. Friedman, The Elements of Statistical Learning, Springer Series in Statistics”, 453-472 Springer [2001].

Clustering based on the K-means algorithm can be used to detect spectral signatures in the image cube. This clustering algorithm classifies clusters maximizing consistency between spectral signatures of the spectral image cube in contrast to conventional K-means clustering algorithms that classify clusters by minimizing Euclidean distances between points. Four different spectral similarity measures including root sum of square error (RSSE, Eq. 1), sum of area difference (SAD, Eq. 2), spectral correlation similarity (SCS, Eq. 3), and spectral angle measure (SAS, Eq. 4) are performed (C.-I. Chang, “An Information Theoretic-Based Approach to Spectral Variability, Similarity and Discriminability for Hyperspectral Image Analysis”, IEEE Trans. Inf. Theory 46(5):1927-1932 [2000]; J. Schwarz J. and K. Staenz, “Adaptive Threshold for Spectral Matching of Hyperspectral Data”, Canadian Journal of Remote Sensing, 27(3): 216-224 [2001]).

$$\begin{matrix}R S S {E}_{orig} = \sqrt{{\sum }_{i = 1}^{N} {( {p}_{i} - {r}_{i} )}^{2}} \\ R S S E = ( R S S {E}_{orig} - m ) / ( M - m ) & [ Equation 1 ] \\ {SAD}_{orig} = {\sum }_{i = 1}^{N} \vert ( {p}_{i} - {r}_{i} ) \cdot {λ}_{inc} \vert \\ SAD = ( {SAD}_{orig} - m ) / ( M - m ) & [ Equation 2 ] \\ SCS = \frac{1}{N - 1} \cdot ( \frac{{\sum }_{i = 1}^{N} ( {r}_{i} - {μ}_{ref} ) ( {p}_{i} - {μ}_{samp} )}{{σ}_{ref} {σ}_{samp}} ) & [ Equation 3 ] \\ SAM = 2 {cos}^{- 1} ( \frac{{\sum }_{i = 1}^{n} {r}_{i} {p}_{i}}{\sqrt{{\sum }_{i = 1}^{N} {r}_{i}^{2}} \sqrt{{\sum }_{i = 1}^{N} {p}_{i}^{2}}} ) / π & [ Equation 4 ]\end{matrix}$$

In the above equations, N represents number of spectra, rᵢ and pᵢ mean intensity of iₜₕ spectrum of the reference signature and the sample pixel signature, λ_{inc} represents increment of the spectra. m and M are the minimum and maximum of RSSE or SAD values respectively. μ_{ref} and σₛₐₘₚ represent mean and standard deviation of reference signature vector and μₛₐₘₚ and σₛₐₘₚ represent those of the sample pixel vector.

Another significant difference between the clustering algorithm in this Example and the conventional K-means algorithm is that the clustering algorithm in this Example uses a threshold similarity index (TSI) and a minimum share of the cluster to determine optimal cluster numbers and remove noise signatures, while the conventional algorithm chooses clusters based on the pre-determined number of clusters (K) which leads to unfavorable results if the data characteristics are unknown.

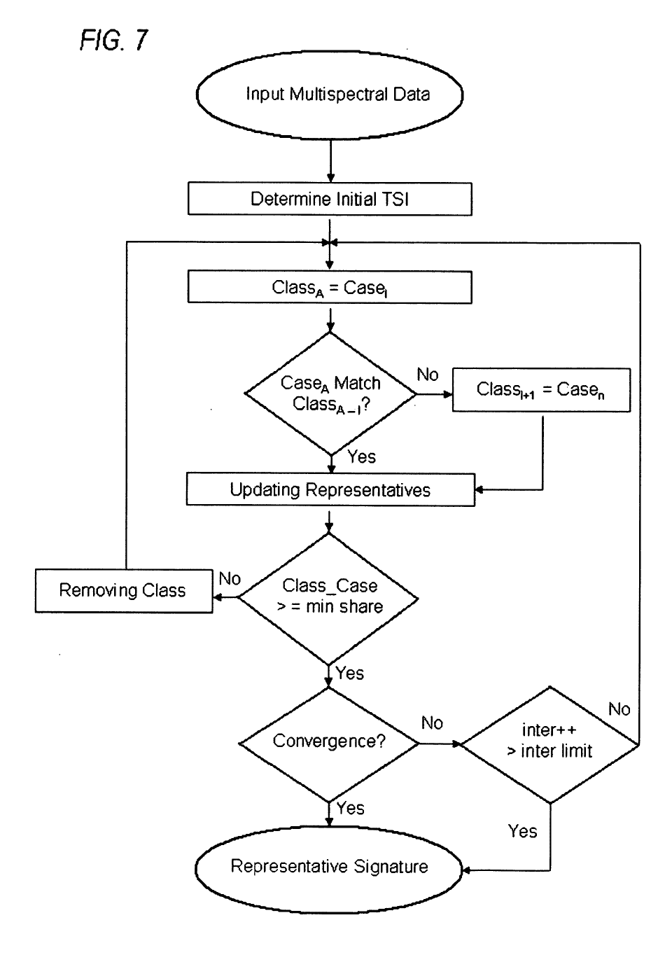

The median (K-means) values of the cluster members are chosen as the cluster representatives. Partitioning spectral data into initial clusters, finding the centroid for each collection, re-partitioning into K clusters from the results and re-finding the centroid are performed repeatedly until relative changes of the total distortion are smaller than threshold values given. The results from those classifiers are reported as spectral signatures clusters and centroid signature from each cluster are used for intelligent imaging process. The flow chart in FIG. 7 shows this process in detail.

After automatic selection of the signatures, the plug-in searches the best matching signature from the in-memory stored signature library and from the local database with pre-determined conditions including species, tissue type, spectral range, and acquisition methods. If the best matched signature was not similar enough (under the limit of consistency input by user), it pops up the window to register the detected signature into the database, and user can determine the name, color allocation, and other conditions in the experiment. If the signature found in the database is well matched with the detected signature, it uses the name and color of the signature found from database.

According to the pre-determined visualization methods including various classification and hybrid color representation schemes, the plug-in displays the result image. If the database is filled with proper signatures, it will display the result image without the need for any signature selection or color allocation processes.

For the evaluation of the usefulness of the intelligent spectral signature imaging without manual spectral signature selection and name/color allocation, a pair of randomly chosen image cubes (1 out of 15 control scanned image cubes, 1 out of 10 aganglionic scanned image cubes) are trained using a modified K-means algorithm with four different spectral signature similarity measures and different TSI values ranging 70 to 90%. Detected signatures are registered into the software database. Evaluation is performed seven times using a different and randomly chosen pair.

Visualization in image space is usually the final step in biomedical applications of spectral imaging. There can be three different strategies to visualize spectral signatures as an image. The most popular one is the classification imaging, where we classify pixels into several groups after matching their spectral signature, and each group is displayed in specified (pseudo) color. Another visualization strategy formulates the image based on calculation of various intensity functions depending on the spectral imaging modes. This can be called quantitative imaging, and shows very fine image detail with flexible modulation capability, although colors in the image are not directly related to tissue identification. Typical examples include narrow-band imaging with custom color bars to visualize certain ranges of spectra. A final strategy is a hybrid of the first two strategies called classification-quantitative hybrid imaging. This approach classifies pixels into several groups using the first strategy, and then calculates intensities for that group using the second strategy. This final strategy is very powerful and purposeful for visualizing data in situ, and shows much improved imaging results in several applications.

### Example 3

A rapid, less invasive, and more accurate technique to diagnose Hirschsprung's disease intraoperatively (i.e., to distinguish aganglionic vs. normal colon) using a hyperspectral optical biopsy (HOB) device according to one embodiment of the invention is tested. This treatment is designed to improve on the existing practice, and can potentially make treatment less expensive.

Prior to performance of the customary pull-through operation, patients (infants and children) who have a new diagnosis of Hirschsprung's Disease will be approached about participating in the study. The diagnosis and treatment plan including operation will be discussed with the parents initially. On a second visit, possible participation in this study will be discussed and study information will be given to parents for reading and discussion with family and friends. If the parent or guardian agree to participate, consent will be obtained.

Once enrolled, the study subject will undergo the pull-through operation. Five (5) laparoscopically procured seromuscular biopsies will be obtained from the colon, identifying the transition point from normal to aganglionic colon. The HOB device will be placed through a laparoscopic port shining light on the colon from 5-10 mm (but not in contact with the tissue) sampling the reflected light (10-15 seconds) at the precise location of the colon identified for biopsy. Each biopsy will be evaluated for presence of ganglion cells using standard methods by a specialist in gastrointestinal pathology. The collected spectral images will be correlated with the pathologic findings for each biopsy site. This will form the basis of a “spectral image library” also known as a “spectral signature library” of normal and aganglionic colon, allowing a testing algorithm to be developed.

For those patients who choose not to participate in the study, the same number of biopsies will be taken as standard of care. Only the hyperspectral imaging of the colon will not be performed.

Once the biopsies are taken and the transition point determined by pathological analysis, the operation is performed in the standard fashion. Routine post-operative care and follow up will be identical for study and non-study patients.

Enrollment will be limited to minors (<18 years of age) since Hirschsprung's Disease presents in infancy and childhood. It would be very rare to have a new diagnosis in adulthood.

Patients with active Hirschsprung's associated enterocolitis (HAEC) will be excluded.

The proposed procedure represents the standard of care in the treatment of Hirschsprung's disease in infancy and childhood. The use of the HOB device will add minimal, if any, time to the entire procedure. As stated prior, once the first seromuscular biopsy is procured and sent to the pathologist for frozen section analysis, there is a waiting period of approximately 60 minutes. During this time the HOB device samplings and seromuscular biopsies will be taken, thereby minimally, if at all, increasing the procedure length.

The post-operative care will be routine care following a pull-through procedure for Hirschsprung's Disease.

A library of in vivo spectral images will be created from both normal and aganglionic bowel in Hirschsprung's Disease patients. The spectral images will be compared and analyzed for reproducible differences that would form the basis of a “testing” algorithm to distinguish normal from aganglionic bowel.

Efficacy will be based on the consistency and reproducibility of the spectral image differences between normal and aganglionic bowel.

The foregoing description of various embodiments of the subject matter known to the applicant at the time of filing this application has been presented and is intended for the purposes of illustration and description. The present description is not intended to be exhaustive nor limit the subject matter to the precise form disclosed and many modifications and variations are possible in the light of the above teachings. The embodiments described serve to explain the principles of the subject matter and its practical application and to enable others skilled in the art to utilize the subject matter in various embodiments and with various modifications as are suited to the particular use contemplated. Therefore, it is intended that the subject matter disclosed herein not be limited to the particular embodiments disclosed.

While particular embodiments of the present subject matter have been shown and described, it will be obvious to those skilled in the art that, based upon the teachings herein, changes and modifications may be made without departing from this subject matter and its broader aspects and, therefore, the appended claims are to encompass within their scope all such changes and modifications as are within the true spirit and scope of this subject matter. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

What is claimed is:

1. A method for treatment of Hirschsprung's disease comprising:
acquiring a multi spectral image of a subject colon;
processing the multi spectral image of the subject colon to develop a digital image; and
analyzing the multi spectral image of the subject colon to differentiate normal from aganglionic colon,
wherein the processing of the multi spectral image is executed in real-time.

2. The method of claim 1, wherein acquisition of the multi spectral image is performed intraoperatively.

3. The method of claim 1, wherein acquisition of the multi spectral image of the subject colon is performed using a hyperspectral optical biopsy device to enhance imagery.

4. The method of claim 1, wherein acquisition of the multi spectral image of the subject colon is achieved using an endoscopic catheter to enhance imagery.

5. The method of claim 1, wherein acquisition of the multi spectral image of the subject colon is achieved using acousto-optic tunable filters (AOTF) to enhance imagery.

6. The method of claim 1, wherein acquisition of the multi spectral image of the subject colon is performed using a laparoscopic adapted emitter-detector probe to enhance imagery.

7. The method of claim 1, wherein processing of the multi spectral image of the subject colon is viewed by a visual imaging output.

8. The method of claim 1, wherein analysis of the multi spectral image of the subject colon comprises establishing a spectral signature for normal and aganglionic colon tissue.

9. The method of claim 1, wherein analysis of the multi spectral image of the subject colon comprises utilizing a machine learned algorithm and a spectral signature database, for automatic spectral signature selection.

10. The method of claim 1, wherein analysis of the multi spectral image of the subject colon comprises utilizing a spectral signature algorithm for differentiation between normal and aganglionic colon tissue.

11. The method of claim 1, wherein analysis of the multi spectral image of the subject colon comprises application of color allocations to differentiate between normal, abnormal and inflamed tissue.

12. The method of claim 1, wherein acquisition of the multi spectral image is performed extralumenally.

13. The method of claim 1, wherein acquisition of the multi spectral image is performed endolumenally.

14. The method of claim 1, wherein acquisition of the multi spectral image is performed in laparoscopic surgery.

15. The method of claim 1, wherein acquisition of the multi spectral image is performed in open surgery.

16. The method of claim 1, wherein visualization of spectral signatures as an image is accomplished by a technique selected from the group consisting of classification imaging, quantitative imaging, and classification-quantitative hybrid imaging.

17. A device for treatment of Hirschsprung's disease, comprising:
a multi spectral imaging mechanism to acquire an image of normal and aganglionic colon;
a multi spectral imaging processor to portray the image in real-time; and
means to analyze and differentiate normal and aganglionic colon in the acquired spectral image.

18. The device for treatment of Hirschsprung's disease of claim 17, wherein the multi spectral imaging mechanism is configured to be used intraoperatively.

19. The device for treatment of Hirschsprung's disease of claim 17, wherein the multi spectral imaging mechanism is selected from the group consisting of a hyperspectral optical biopsy device, an endoscopic catheter, an acousto-optic tunable filter (AOTF), and a laparoscopic adapted emitter-detector.

20. The device for treatment of Hirschsprung's disease of claim 17, wherein the multi spectral imaging processor utilizes spectral signatures to differentiate between normal and aganglionic colon tissue.

21. The device for treatment of Hirschsprung's disease of claim 20, wherein the multi imaging processor utilizes a machine learned algorithm and a spectral signature database, for automatic spectral signature selection.

22. The device for treatment of Hirschsprung's disease of claim 21, wherein the automatic spectral signature selection utilizes plug-in software, database management software and algorithm calculation software.

23. The device for treatment of Hirschsprung's disease of claim 20, wherein the automatic spectral signature selection is based on a K-means algorithm for unsupervised learning or indirect knowledge discovery.

24. The device for treatment of Hirschsprung's disease of claim 17, wherein the multi spectral imaging processor comprises a visual imaging output.

25. The device for treatment of Hirschsprung's disease of claim 17, wherein the means to analyze and differentiate an image of normal and aganglionic colon is a function of the spectral statistics associated with normal and aganglionic colon.

26. The device for treatment of Hirschsprung's disease of claim 20, wherein visualization of spectral signatures is accomplished by a technique selected from the group consisting of classification imaging, quantitative imaging and classification-quantitative hybrid imaging.

27. A device for treatment of Hirschsprung's disease, comprising:
an endoscopic probe comprising an emitter to produce a signal, and a detector for collecting a refracted signal;
a processor for analyzing the refracted signal from the detector, recognizing normal and aganglionic colon tissue, and identifying normal and aganglionic colon tissue in real-time; and
an energy source providing power to the device.

28. The device for treatment of Hirschsprung's disease of claim 27, wherein the processor employs spectral signatures to differentiate between normal and aganglionic colon tissue.

29. The device for treatment of Hirschsprung's disease of claim 27, wherein the processor utilizes a machine learned algorithm and a spectral signature database, for automatic spectral signature selection.

30. The device for treatment of Hirschsprung's disease of claim 29, wherein the automatic spectral signature selection utilizes plug-in software, database management software and algorithm calculation software.

31. The device for treatment of Hirschsprung's disease of claim 30, wherein the automatic spectral signature selection is based on a K-means algorithm for unsupervised learning or indirect knowledge discovery.

32. The device for treatment of Hirschsprung's disease of claim 27, wherein the process for analyzing, recognizing, and identifying an image of normal and aganglionic colon is a function of the spectral statistics associated with normal and aganglionic colon.

33. The device for treatment of Hirschsprung's disease of claim 27, wherein the process for identifying normal and aganglionic colon utilizes a visual imaging output for displaying a colon tissue image.

34. The device for treatment of Hirschsprung's disease of claim 33, wherein the process of producing the colon tissue image is accomplished by a technique selected from the group consisting of classification imaging, quantitative imaging and classification-quantitative hybrid imaging.

35. A device for treatment of Hirschsprung's disease in an individual, comprising:
a laparoscopic adapted probe;
an emitter connected to the laparoscopic adapted probe, wherein the emitter produces a signal;
a detector connected to the laparoscopic adapted probe, wherein the detector collects a refracted signal produced by the emitter and reflected by the colon tissue in the individual;
a processor for analyzing the refracted signal from the detector, recognizing normal and aganglionic colon tissue, and producing an image of the colon tissue;
a visual imaging output for displaying the image in real-time; and
an energy source for providing power to the device.

36. The device for treatment of Hirschsprung's disease of claim 35, wherein the processor utilizes spectral signatures to differentiate between normal and aganglionic colon tissue.

37. The device for treatment of Hirschsprung's disease of claim 35, wherein the processor utilizes a machine learned algorithm and a spectral signature database, for automatic spectral signature selection.

38. The device for treatment of Hirschsprung's disease of claim 37, wherein the automatic spectral signature selection utilizes plug-in software, database management software and algorithm calculation software.

39. The device for treatment of Hirschsprung's disease of claim 37, wherein the automatic spectral signature selection is based on a K-means algorithm for unsupervised learning or indirect knowledge discovery.

40. The device for treatment of Hirschsprung's disease of claim 35, wherein the process for analyzing and recognizing an image of normal and aganglionic colon is a function of the spectral statistics associated with normal and aganglionic colon.

41. The device for treatment of Hirschsprung's disease of claim 35, wherein the process of producing the colon tissue image is accomplished by a technique selected from the group consisting of classification imaging, quantitative imaging and classification-quantitative hybrid imaging.

42. A computer-usable medium having readable instructions stored thereon for execution by a processor to perform a method comprising:
obtaining spectral images of a patient's colon;
channeling the spectral images through an interferometer to produce a spectral signature for each image;
analyzing the spectral signatures for each spectral image; and
identifying variations in the spectral signatures for normal and aganglionic colon tissue.

43. The method of claim 42, further comprising digital image processing software for spectral signature analysis.

44. The method of claim 42, further comprising digital image processing software for identifying variations in the spectral signature for normal and aganglionic colon tissue.

45. The method of claim 42, wherein the spectral signature of normal and aganglionic colon are converted into a series of TIFF format images.

46. The method of claim 45, wherein the series of TIFF format images of normal and aganglionic colon are imported into database management software for registration of spectral signatures for normal and aganglionic colon tissue.

47. The method of claim 42, further comprising application of a data mining algorithm for unsupervised learning or indirect knowledge discovery of the spectral signatures.

48. The method of claim 42, wherein a second spectral signature may be contrasted with spectral signatures in the database using the digital imaging processing software and the data mining algorithm.

49. The method of claim 42, wherein the identified variations in the spectral signatures for normal and aganglionic colon tissue are designated colors to specific spectral signatures.

50. The method of claim 42, wherein the identified variations in the spectral signatures for normal and aganglionic colon tissue are visualizing in image space by a technique selected from the group consisting of classification imaging, quantitative imaging and classification-quantitative hybrid imaging.
